(19) United States
(12) Patent Application Publication
(10) Pub. No.: US 2023/0259673 A1
(43) Pub. Date: Aug. 17, 2023

(54) QUANTUM COMPUTER-IMPLEMENTED SOLVER

(71) Applicant: The University of Melbourne, The University of Melbourne, Victoria (AU)

(72) Inventors: Lloyd Christopher Leonard Hollenberg, Melbourne (AU); Charles David Hill, Melbourne (AU); Michael Alexander Jones, Melbourne (AU); Harish James Vallury, Melbourne (AU); Aochen Duan, Melbourne (AU); Sarama Usriya Yi Xiang Tonetto, Melbourne (AU)

(21) Appl. No.: 18/011,174

(22) PCT Filed: Jun. 25, 2021
(86) PCT No.: PCT/AU2021/050674

§ 371 Date: Dec. 17, 2022

(30) Foreign Application Priority Data
Jun. 26, 2020 (AU) 2020902156

(51) Int. Cl. G06F 30/20; G06N 10/40; G06N 10/20

(52) U.S. Cl. CPC G06F 30/20; G06N 10/40; G06N 10/20; G06F 2111/10

(57) ABSTRACT

This disclosure relates to a method for estimating a solution to a problem represented by a Hamiltonian on a quantum computer having a quantum state. A classical computer determines a trial state and evolves the quantum state of the quantum computer based on the trial state. The computer then determines estimates for expectation values with respect to the trial state of powers of the Hamiltonian based on measurements from the quantum computer and calculates an estimate of the solution based on the estimates for the expectation values of powers of the Hamiltonian for the trial state. The computer then repeated updates the trial state and repeats the measuring steps to iteratively improve the estimate of the solution.

### TECHNICAL FIELD

This disclosure relates to solving problems, including but not limited to mathematical optimisation problems, molecular chemistry problems and physics problems, on quantum computers.

### BACKGROUND

Quantum computers (QCs) have generated significant excitement in scientific as well as commercial communities as the technology gradually matures. However, at the current state, one major drawback of quantum computers is that the quantum information processing can suffer errors due to interactions with the environment and/or control errors in the implementation of quantum logic gates. These errors mean that quantum information is often lost before the required sequence of quantum logic operations in a given quantum algorithm can be completed. In other words, current quantum computers can only process relatively ‘short’ sequences of quantum logic operations, which means the application to useful problems remains limited.

Promising applications of existing quantum computers are in finding solutions to problems that can be cast into a qubit representation. For example, quantum computers can be used to study the behaviour of electrons in a molecule and corresponding orbitals. This can then be used to predict the efficacy of drugs or support the discovery of new materials. In a simplified way, this problem is an energy minimisation problem because electrons would typically ‘fall’ into the lowest possible energy state. Therefore, once the energy in a molecule can be expressed in terms of qubit observations, the energy can be minimised iteratively by changing input parameters describing the electron configuration. The lowest energy that can be found as a result of the qubit operations, should then correspond to the energy, and therefore the electron configuration, in the molecule under study. Many problems in optimisation can be cast into an effective energy minimisation problem.

An example of how to approach such problems on a quantum computer is generally described as the Variational Quantum Eigensolver (VQE) as, for example, in A. Peruzzo, J. McClean et. al., A variational eigenvalue solver on a photonic quantum processor, Nat. Commun., 2014, doi:10.1038/ncomms5213, which is incorporated herein by reference. In this approach, a classical computer performs an optimisation routine to minimise the measured energy with respect to a given trial state. Further, a quantum computer is configured to represent the trial state and compute the energy in the problem at hand.

FIG. 1 illustrates a VQE 100 comprising a quantum computer 101 and a classical computer 102. The quantum computer 101 comprises a number of qubits 103 (shown as black dots), a trial state controller 104 and a measurement unit 105. In a simplified view, the measurement unit 105 applies operations to the qubits 103 so that measurements over the collective can be related to the problem to be solved. More particularly, when individual qubits are observed, they provide one of two possible states (similar to a digital computer). However, the qubit may have been in a quantum superposition of two possible states and the measurement of the quantum superposition collapses it into one of the pure states according to the underlying probability distribution of the quantum superposition. The qubits in 103 may have been in a collective quantum superposition of all possible states and the measurement of the collective quantum superposition collapses it according to the underlying probability distribution of the collective quantum superposition which is related to the problem to be solved. This means that if the same observation is repeated many times, all possible states may be observed in different proportion. Since the problem to be solved remains the same, and is encoded in the trial state represented in the combined quantum state of the qubits the measurement unit 105 applies the same operations of the qubits at each repetition to obtain multiple sampled measurements, such as the underlying statistical distribution. Classical computer 102 can then calculate an energy estimate from the summation of multiple sampled measurements for each of the energy parts.

The trial state is governed by parameters that are changed in order to minimise the measured energy. Classical computer 102 determines an initial trial state and controls the trial state controller 104 accordingly. Classical computer 102 then enters a sampling loop 110 in order to collect a number of measurements from the measurement unit 105 and calculate an energy estimate for the initial trial state. Once the sampling loop is finished, that is, a sufficient number of samples is collected for each term in the Hamiltonian, classical computer 102 computes the energy of the current trial state and updates the trial state in a minimisation loop over the parameters in the trial state using minimisation routines 111. The new trial state is then sampled again through sampling loop 110. If the new energy estimate is higher, the direction of the trial state parameter flow is reversed. If the energy estimate is lower, the new trial state is accepted and further adjusted in the next round, and so on.

It is noted that each round of trial state preparation requires a number of operations to be performed on the qubits 103. The number of operations may be low, which is referred to a shallow trial state circuit or may be high, which is referred to a deep trial state circuit. The space of all possible trial states can be considered a search space in which to find an optimal solution for the energy. If the search state is reduced, it is possible that the global optimum is not in the reduced search space. This means that reducing the search space may result in a less accurate solution or overestimation of the actual minimal energy.

Unfortunately, current quantum computers do not provide the qubit stability required for deep trial state circuits. As a result, the search space of the VQE is limited to short trial state circuits. This generally results in over-estimations of the energy.

FIG. 2 illustrates how the complexity of the trial state is systematically increased in the VQE algorithm as it would be implemented on a quantum computer. The estimated energies gradually converge to the exact value indicated at 201, at the cost of implementing increasingly deep circuits and including more trial state parameters in the classical optimisation loop.

### SUMMARY

This disclosure provides an improvement to the variational quantum eigensolver. This improvement modifies the measurement unit 105 to capture higher order measurements. While the higher order measurements use additional information, an improved accuracy in the energy estimate can be achieved using shorter trial states.

There is provided a method for estimating a solution to a problem represented by a Hamiltonian on a quantum computer (digital or otherwise) having a quantum state. The method comprises:

determining a trial state and evolving the quantum state of the quantum computer based on the trial state;

determining estimates for expectation values with respect to the trial state of powers of the Hamiltonian based on measurements from the quantum computer;

calculating an estimate of the solution based on the estimates for the expectation values of powers of the Hamiltonian for the trial state; and

repeatedly updating the trial state and repeating the measuring steps to iteratively improve the estimate of the solution.

The solution to the problem may be an energy state of a physical system represented by the Hamiltonian on a quantum computer.

The energy state may comprise one or more of a ground state and an excited state.

The solution to the problem may be a configuration of the physical system.

The solution to the problem may be an expectation value of a quantity with respect to the configuration of the system.

The solution may be a wavefunction of the physical system.

Calculating the estimate of the solution may comprise calculating more than first order moments of the Hamiltonian based on the multiple samples and calculating the estimate of the solution based on the more than first order moments.

Calculating the estimate of the solution may be based on cumulants that represent connected moments.

The cumulants may correspond to values required for a Lanczos method.

The Lanczos method may be based on parameters of an approximate representation of the Hamiltonian on a basis of the trial state and calculating the estimate of the solution based on the parameters of the approximate representation of the Hamiltonian.

The approximate representation of the Hamiltonian may comprise a tri-diagonal matrix.

The cumulants may be based on a binomial transformation of the more than first order moments.

The trial state may correspond to a ground-state or excited states of the Hamiltonian.

The trial state may comprise one or more adjustable parameters and updating the trial state comprises updating the one or more adjustable parameters to iteratively improve the estimate of the solution.

The method may further comprise:

representing the Hamiltonian as a combination of multiple first order Pauli strings and the powers of the Hamiltonian as multiple, more than first order Pauli strings;

measuring an output corresponding to the multiple first order Pauli strings of the quantum computer as an evolution of the trial state, to obtain first order measurements;

measuring the output corresponding to the multiple, more than first order Pauli strings of the quantum computer as an evolution of the trial state to obtain multiple, more than first order measurements; and

repeating the steps of measuring the output of the quantum computer to obtain the multiple samples of the first order measurements and the more than first order measurements for determining the estimates for the expectation values.

Determining the multiple, more than first order Pauli strings may comprise determining multiples of the multiple first order Pauli strings.

Measuring the output corresponding to the multiple, more than first order Pauli strings may comprise applying quantum operations of the multiple, more than first order Pauli strings to the quantum computer.

Determining the multiple, more than first order Pauli strings may comprise combining elements in the multiple, more than first order Pauli strings into equivalent elements to reduce the multiple, more than first order Pauli strings.

Combining the elements may comprise determining tensor product basis sets of Pauli strings that mutually qubit-wise commute.

Measuring the output corresponding to the multiple, more than first order Pauli strings may comprise measuring only the tensor product basis sets.

The Hamiltonian may be associated with a parameter or set of parameters that is included in the multiple first order Pauli strings and the multiple, more than first order Pauli strings; and the method may comprise minimising the parameter.

The parameter or set of parameters is associated with explicit quantum-mechanical term or terms representing a dynamical quantity to be determined with respect to the problem.

There is further provided a system for estimating a solution to a problem represented by a Hamiltonian on a digital quantum computer. The system comprises:

multiple qubits;

a trial state controller to set the multiple qubits into a trial state;

a read-out component to measure a quantum state of the qubits; and

a classical computer to control the trial state controller and the read-out component, the classical computer being configured to:
determine a trial state and initialising the trial state on the quantum computer;
determine estimates for expectation values of powers of the Hamiltonian based on multiple samples measured from the quantum computer encoding the trial state;
calculate an estimate of the solution based on the estimates for the expectation values of powers of the Hamiltonian for the trial state; and
repeatedly update the trial state and repeat the measuring steps to iteratively improve the estimate of the solution.

### BRIEF DESCRIPTION OF DRAWINGS

FIG. 1 illustrates a Variational Quantum Eigensolver according to the prior art.

FIG. 2 schematically illustrates how ground state energy estimates are determined according to the prior art.

An example will now be described with reference to the following drawings:

FIG. 3 illustrates a moments-based quantum solver (MBQS).

FIG. 4 illustrates Pauli strings of different order corresponding to powers of the Hamiltonian describing the problem at hand.

FIG. 5 illustrates the Lanczos method for computing the ground-state of a Hamiltonian system based on recursive transformation to tri-diagonal form {αₚ, βₚ}, and subsequent diagonalization of truncated matrices converging to the ground-state energy. Even for large systems, convergence is rapid, i.e. at pₘₐₓ<<2ⁿ.

FIG. 6 illustrates the approximate Lanczos system obtained from truncating the maximal Hamiltonian moment with respect to a given trial state used to determine an approximation to the tri-diagonal system. Diagonalisation of the full approximated system results in better estimates than the variational calculation (equivalent to truncating to [symbol] H¹ [symbol] ).

FIG. 7 illustrates hybrid quantum algorithmic approaches to energy determination/optimisation problems—VQE and QAOA. These approaches generally use relatively long circuits and may suffer from catastrophic error accumulation.

FIG. 8 illustrates a short-depth hybrid quantum algorithm to compute Hamiltonian moments to obtain the Lanczos energy estimate, E_{L}. The trial state parameter “a”, may represent a set or vector of parameters. It is shown for 1 iteration, but may be extended to more than 1 iteration with more parameters to obtain higher precision.

FIG. 9 provides a demonstration of the moment method on the D-dimensional Heisenberg model—a quantum spin model analogous to problems translated to a qubit system on a QC. The first order Lanczos estimate consistently provides a significant correction to the trial-state energy and already gets very close to the exact answer (where known).

FIG. 10 illustrates an example flow of the method disclosed herein. For problems requiring determination of non-energy quantities the “quantum spark” may be designed to produce these quantities in the zero limit.

FIG. 11 illustrates how the moments can be used to improve on the trial state energy. In particular, FIGS. 11 shows the various ground state energy estimates for a 6-site Heisenberg antiferromagnet, with [symbol] H [symbol] (prior art, dashed lines) and E_{L} (this disclosure, solid lines). Each vertical panel corresponds to a different trial state. The exact ground state energy is marked as a line through all panels at −12.5 for comparison.

FIG. 12a illustrates a method for estimating a solution to a problem represented by a Hamiltonian on a quantum computer. FIG. 12b illustrates another method for estimating a solution to a problem represented by a Hamiltonian on a quantum computer using higher order Pauli strings.

FIGS. 13a-13c illustrate an overview of Hamiltonian exponentiation. FIG. 13a provides a grouping of concatenated Pauli strings [Rⱼ⁽ⁿ⁾] in Hⁿ into Tensor Product Basis (TPB) sets [Qₖ⁽ⁿ⁾]. FIG. 13b illustrates an example: the first few TPB sets for the q=6 2D Heisenberg model. FIG. 13c illustrates operator term counts for H⁴ before and after TPB grouping for the quadratic model defined on a 1D chain, heavy-honeycomb and square lattice.

FIG. 14 illustrates trial-state circuit construction. (a) General multi-parameter p-block trial-state construction for hybrid variational approaches. (b) Quantum circuits for initialising the trial-state |ϕₜᵣᵢₐₗ(θ) [symbol] over a 1D qubit array for the 2D 2×3 problem: θ=π (Néel state) and θ=0.7π. Vertical bar at right indicates the level of bi-partite entanglement-entropy in the (ideal) trial-state at 1-bit. Probability distributions are shown together with the overall phase of the individual state amplitudes (visualisation using the quantum user interface (QUI) system [34])

FIG. 15 illustrates the moments-based quantum computing algorithm applied to the q=6 2D Heisenberg model (uniform couplings) with respect to a single θ trial-state (FIG. 14). (a) Quantum computed Hamiltonian moments and cumulants, respectively, assembled from the measurement of the expectation values of the TPB Pauli strings [symbol] [Qₖ⁽ⁿ⁾] [symbol] . Data points correspond to calculations on a physical quantum processor with statistical error bars corresponding to 5×1024 shots per expectation value. Solid grey lines are simulations carried out using the Qiskit QASM simulator at zero noise. (b) Quantum processor device map for showing the qubits used in the computation. (c) QCM infimum estimate obtained from the device runs (bottom data points), plotted as a function of the trial-state parameter θ, directly compared with the benchmark variational results derived from [symbol] H [symbol] _{θ} (top data points). The exact value is shown as a dashed line. Different solid lines correspond to simulations at different noise levels, from zero to a high-noise scenario in steps of 2× the default device error model.

FIG. 16 illustrates a comparison of QCM infimum and variational benchmark estimates for the generalised 2D Heisenberg model on square lattices of increasing size. (a) Experimental results obtained from the physical quantum devices device1 and device2 (device2 data for 4×4: θ=0.7π and θ=1.3π), with 8192 shots per point, plotted as a function of θ. Herein, device1 and device2 are the IBM Quantum devices ibmq_manhattan and ibmq_toronto, respectively. Solid lines correspond to zero-noise simulations (Qiskit QASM simulator) for variational and infimum estimates. The exact ground-state energy is plotted as a green dashed line. For each lattice size the trial state |ϕₜᵣᵢₐₗ(θ) [symbol] (FIG. 15(b)) was encoded using a linear chain of qubits, as shown in the device map (b). (c) Approximation ratios with respect to the exact results for the uniform coupling model and the random coupling ensemble average (10³ instances). (d) Frequency distribution over the random coupling ensemble for QCM infimum estimates and variational benchmark, compared to the exact results calculated for 3×3 and 4×4.

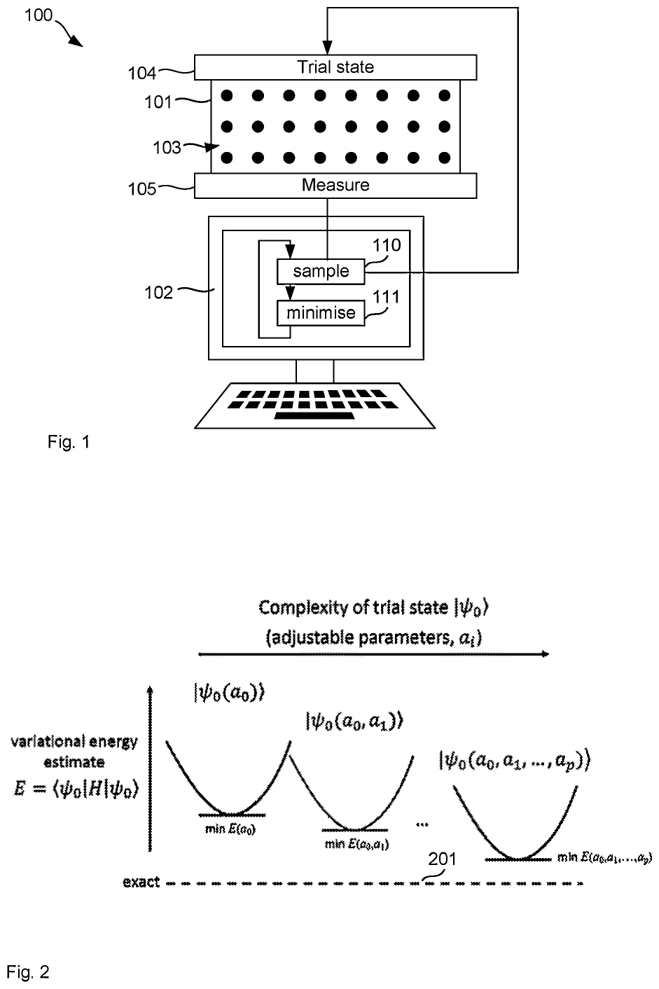

### DESCRIPTION OF EMBODIMENTS

### Digital Quantum Computer

There are currently two main approaches to physically implementing a quantum computer: analog and digital. Analog approaches are further divided into quantum simulation, quantum annealing, and adiabatic quantum computation. Digital quantum computers use quantum logic gates to do computation. This disclosure relates to quantum computers (digital or analog/adiabatic), where each of the qubits 103 of quantum computer 101 and their interactions is controlled via the application of digital quantum logic. This is distinct to a “quantum annealer” based on controlled adiabatic evolution of the system.

### Moments-Based Quantum Solver

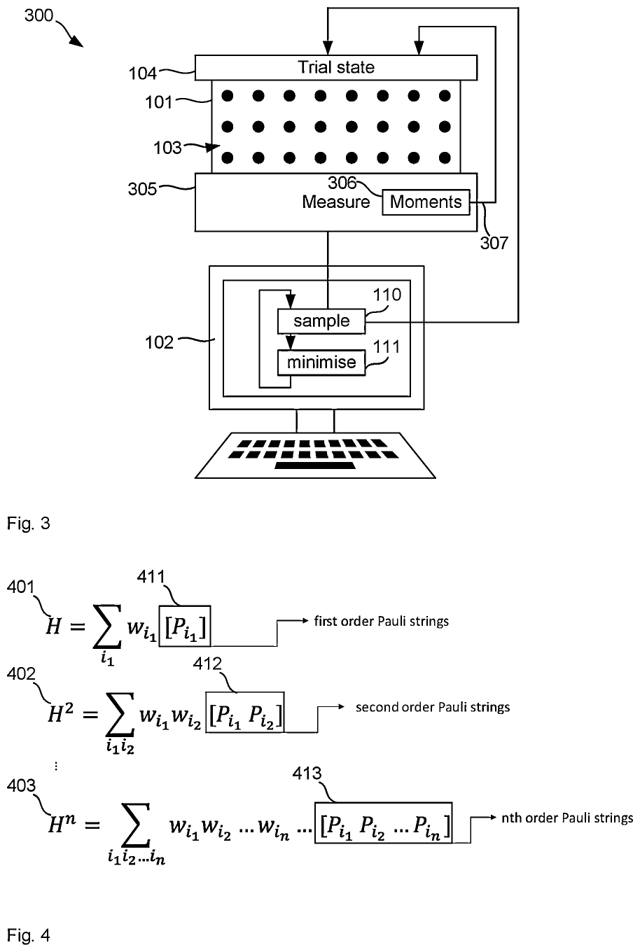

FIG. 3 illustrates a moments-based quantum solver (MBQS) 300. Similar to VQE 100 in FIG. 1, MVQE 300 comprises a quantum computer 101, classical computer 102, qubits 103, and a trial state controller 104. In contrast to FIG. 1, the measurement unit 305 now includes a moment generator 306. It is noted that moment generator 305 is shown to be part of the measurement unit 305 but may equally be integrated into software executed on classical computer 102.

Essentially, moment generator 306 adds another loop 307 to calculate multiple, higher order moments of the measurements for each sample 110. This captures the dynamics of the system at the measurement instead of using deeper trial states, at the expense of an increased amount of measurements.

### Pauli Strings

Further information on Pauli Measurements can be found, for example, in the documentation of the Microsoft Q# language under “Pauli measurements” (https://docs.microsoft.com/en-us/quantum/concepts/pauli-measurements), which is included herein by reference. Mathematically, the multiple higher order moments can be represented by higher order Pauli strings. A higher order Pauli string, or synonymously a more than first order Pauli string, as the term is used herein, is a Pauli string that is the result of a product of two or more first order Pauli strings or other higher order Pauli strings. So for example, a given Hamiltonian that captures the set-up of the system under investigation, can be represented as its first order moment H, which is constructed of a sum of weighted Pauli strings Pᵢ. These Pauli strings are referred to as first order Pauli strings:

$$H = {\sum }_{i} {w}_{i} {P}_{i} .$$

In addition to this first order moment H, measurement unit 305 measures more than first order moments H², H³, and so on. This results in a product of Pauli strings, which is another, more than first order Pauli string. These higher order product Pauli strings can be reduced to “reduced Pauli strings” R (1004) over the commensurate number of qubits in the quantum computer. So [symbol] H² [symbol] can also be represented as a sum over weighted Pauli strings and after reduction can be evaluated (i.e. measured) in the same way as [symbol] H [symbol] through repeated intialisation and sampling of the Pauli string in question over the final state of the quantum computer.

FIG. 4 illustrates Pauli strings of different order. In particular, a first order moment 401 comprises first order Pauli strings 411, a second order moment 402 comprises second order Pauli strings 412 and an nth order moment 403 comprises nth order Pauli strings 413. These are reduced to reduced Pauli strings R (1004) of length commensurate with the number of qubits in the system, and measured as described above.

There may be a number of different ways of using the higher order moments for a given Hamiltonian determined on the quantum computer (e.g. Lanczos method, projector Monte-Carlo, partition function determination, cluster expansions).

Generally, these involve computing expectation values of quantities [symbol] ψ₀|HⁿAHⁿ|ψ₀ [symbol] for a given operator quantity A and trial state |ψ₀ [symbol] . One approach is the Lanczos method, which approximately diagonalises a large dimension Hamiltonian by iteratively constructing a tri-diagonal matrix. The Lanczos method iteratively constructs a tri-diagonal matrix by choosing a starting state, |ψ₁ [symbol] =|ψ₀ [symbol] , acting on it with the Hamiltonian and defining the orthogonal component of the state produced as |ψ₂ [symbol] :

H|ψ₁>=α₁|ψ₁>+β₁|ψ₂>

The process is applied recursively,

H|ψᵢ>=αᵢ|ψᵢ>+βᵢ|ψᵢ₊₁>+βᵢ₋₁|ψᵢ₋₁>, α₀=β₀=0

and the values of αᵢ and βᵢ are used to construct a tri-diagonal approximate representation of the Hamiltonian on the basis, {|ψᵢ>},

$${H}_{M} = ( \begin{matrix}{α}_{1} & {β}_{1} & & & \\ {β}_{1} & {α}_{2} & {β}_{2} & & \\ & {β}_{2} & {α}_{3} & {β}_{3} & \\ & & ⋱ & ⋱ & ⋱ \\ & & & {β}_{M - 1} & {α}_{M}\end{matrix} )$$

where M is the truncation order of the tri-diagonal matrix. The values of αᵢ and βᵢ can be related to the moments of the Hamiltonian For example,

$$\begin{matrix}〈 {ψ}_{1} \vert H \vert {ψ}_{1} 〉 & = & {α}_{1} , \\ 〈 {ψ}_{1} \vert {H}^{2} \vert {ψ}_{1} 〉 & = & {α}_{1}^{2} + {β}_{1}^{2} , \\ 〈 {ψ}_{1} \vert {H}^{3} \vert {ψ}_{1} 〉 & = & {α}_{1}^{3} + 2 {α}_{1} {β}_{1}^{2} + {α}_{2} {β}_{1}^{2} , \\ & ⋮ & \end{matrix}$$

Inverting these equations allows calculation of the tri-diagonal matrix from the Hamiltonian moments. The eigenvalues of the tri-diagonal matrix rapidly approach those of the full Hamiltonian as the truncation order, M, increases. Diagonalisation of the smaller matrix, H_{M}, can then be performed numerically. Through the introduction of the ‘plaquette expansion’ one particular general analytical form was obtained which may prove useful in the quantum computing context. Namely, the Lanczos procedure gives a non-trivial correction to the variational method as:

$${E}_{L} = {c}_{1} + \frac{{c}_{2}^{2}}{{c}_{2} {c}_{4} - {c}_{3}^{2}} ( \sqrt{3 {c}_{3}^{2} - 2 {c}_{2} {c}_{4}} - {c}_{3} )$$

where the connected moments are given by

$${c}_{i} = {〈 {H}^{i} 〉}_{c} = 〈 {H}^{i} 〉 - {\sum }_{p = 0}^{i - 2} ( \begin{matrix}i - 1 \\ p\end{matrix} ) {〈 {H}^{p + 1} 〉}_{c} 〈 {H}^{i - p - 1} 〉 ,$$

so c₁=<H>=E_{VQE}.

The correction to the variational estimate of the ground state energy (i.e. E_{VQE}=c₁) given by the above equation is generally more accurate for a well-chosen trial state. The Lanczos method is able to find an accurate approximation to the ground state energy, even when the trial state does not have perfect overlap with the true ground state. As such its use with the VQE algorithm may allow simpler trial states to give reasonably accurate results allowing for decreased circuit depths, better accuracy and potentially application to larger problems for which the noise level might otherwise prevent construction of trial states. It is noted, however, that other methods than the Lanczos method can be used for finding an accurate approximation to the ground state energy based on higher-order moments, such as the connected moments expansion, Quantum Monte Carlo, and minimisation based on finite moments orders.

The Lanczos method computes the low lying states of a Hamiltonian H expressed in terms of a matrix eigenvalue problem in convenient basis. Computer 102 starts from a well-chosen trial-state |ψ₀ [symbol] (which may be a variational state), and the Hamiltonian matrix is iteratively transformed into tri-diagonal form (Lanczos basis, {|ψₚ [symbol] }) with diagonal and off-diagonal elements αₚ and βₚ respectively. The transformation is very efficient—e.g. for a system of n spin ½ particles with Hilbert space dimension 2ⁿ, the convergence to the ground state energy is rapid and typically one obtains excellent results for a truncated tri-diagonal system pₘₐₓ<<2ⁿ.

### Measuring the Hamiltonian Moments on a Quantum Device

To apply the correction given by the equation above for E_{L} in the context of quantum computation, system 300 uses a method of measuring the Hamiltonian moments, <H^{p}>. Given a Hamiltonian written as a sum of weighted Pauli strings

$$H = {\sum }_{i} {w}_{i} {P}_{i}$$

H² can be written as,

$${H}^{2} = {\sum }_{i} {\sum }_{j} {w}_{i} {w}_{j} {P}_{i} {P}_{j} .$$

Since the products of Pauli matrices are:

X·Y=−Y·X=Z,

Y·Z=−Z·Y=X,

Z·X=−X·Z=Y

and X·X=Y·Y=Z·Z=I,

the product of two Pauli strings is another Pauli string—by such reductions and their generalisations we arrive at a set of reduced Pauli strings, R. So <H²> can also be represented as a sum over weighted reduced Pauli strings and so can be evaluated in the same way as <H>. This argument is applied systematically to allow evaluation of moments up to <H⁴>. Using these moments, system 300 can implement the moments-based correction on quantum devices, such as qubits 103.

### Quantum Computing and the Lanczos Moment Expansion

System 300 exploits the connection between the Lanczos method and moments of the Hamiltonian with respect to the trial-state, [symbol] Hⁿ [symbol] = [symbol] ψ₀|Hⁿ|ψ₀ [symbol] . The moment formalism enables both connecting to QC, and also encapsulating higher order dynamics of the system in these moments to obtain corrections to the variational result without increasing the quantum circuit depth.

Starting from the trial state, |ψ₀ [symbol] , one can follow the Lanczos recursion analytically in ket form from |ψ₁ [symbol] =|ψ₀ [symbol] to construct the tri-diagonal basis {|ψₚ [symbol] }: βₚ₋₁|ψₚ [symbol] =(H−αₚ₋₁)|ψₚ₋₁ [symbol] −βₚ₋₂|ψₚ₋₂ [symbol] . The Lanczos tri-diagonal elements can be written in terms of Hamiltonian moments of increasing order, i.e. αₚ=αₚ( [symbol] H¹ [symbol] , . . . , [symbol] H^{2p-1} [symbol] ) and βₚ=βₚ( [symbol] H¹ [symbol] , . . . , [symbol] H^{2p} [symbol] ). It is noted that in some examples, the expressions for the αₚ and βₚ become complicated, and Hamiltonian moments are classically difficult to compute in general.

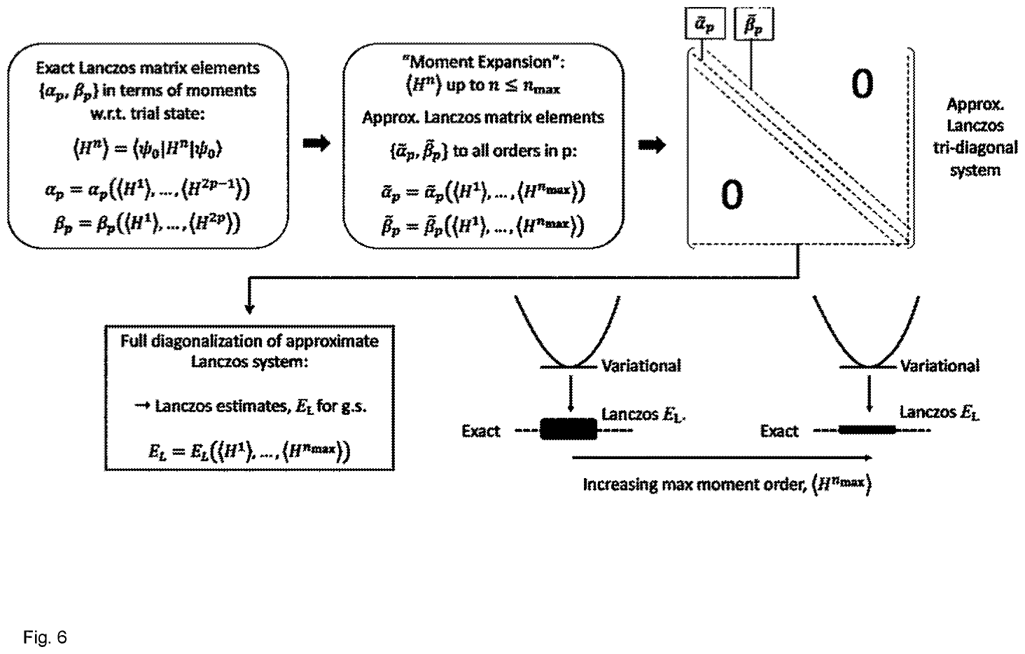

One step in connecting Hamiltonian moments with the Lanczos procedure in a practical way is the realization that the Lanczos elements αₚ and βₚ could be recast analytically for any iteration p as an expansion in the Hamiltonian moments of increasing order. Based on this expansion (“plaquette expansion”) for αₚ and βₚ, the full Lanczos system can be approximated to finite orders in the moments, i.e. [symbol] Hⁿ [symbol] up to n≤nₘₐₓ, and systematic estimates of the ground-state energy based on the trial-state could be obtained by diagonalisation of the approximated system as shown in FIG. 6.

### Analytic First Order Lanczos Energy Estimate

A particularly convenient and useful general result can be obtained at first order in the plaquette expansion, corresponding to retaining the first four Hamiltonian moments: [symbol] H¹ [symbol] , . . . , [symbol] H⁴ [symbol] . The moments build in higher order dynamics into the trial-state, |ψ₀ [symbol] , and hence the dramatically improved quality of the energy estimate beyond the variational result which corresponds to the zeroth order [symbol] H¹ [symbol] . However, there is a certain amount of redundancy in the moments, and it is possible to more efficiently encapsulate the dynamics in “connected moments” (cumulants) [symbol] Hⁿ [symbol] _{c}, obtained through a binomial transformation:

$${〈 {H}^{n} 〉}_{c} = 〈 {H}^{n} 〉 - {\sum }_{p = 0}^{n - 2} ( \begin{matrix}n - 1 \\ p\end{matrix} ) {〈 {H}^{p + 1} 〉}_{c} 〈 {H}^{n - 1 - p} 〉$$

In a quantum field theory this transformation removes irrelevant disconnected Feynman diagrams, in this QC context extraneous information is similarly removed. In what follows this disclosure will use the notation: cₙ= [symbol] Hⁿ [symbol] _{c}. Based on orthogonal polynomial theory, the full Lanczos system truncated to [symbol] H⁴ [symbol] can be diagonalized analytically to produce a moment-based Lanczos E_{L}(c₁ . . . c₄) estimate to the ground-state energy from a given trial-state:

$${E}_{L} = {c}_{1} + \frac{{c}_{2}^{2}}{{c}_{2} {c}_{4} - {c}_{3}^{2}} ( \sqrt{3 {c}_{3}^{2} - 2 {c}_{2} {c}_{4}} - {c}_{3} )$$

This result includes dynamically driven corrections to 4th order in the moments on the variational result c₁= [symbol] H¹ [symbol] _{c}= [symbol] H¹ [symbol] for a given trial state (equivalent to summing over classes of diagrams in a quantum field theory context).

The (classical) difficulty of the computation of the moments themselves depends on the type of problem in question: for homogenous systems (e.g. all parts uniformly coupled) one can analytically derive expressions for [symbol] Hⁿ [symbol] , even when the number of parts becomes infinite However, the interesting cases derived from real-world problems generally involve heterogenous couplings and for these systems the whole Hilbert space of dimension 2ⁿ comes into play—an ideal problem to set a quantum computer on.

### Moment Based Quantum Computation

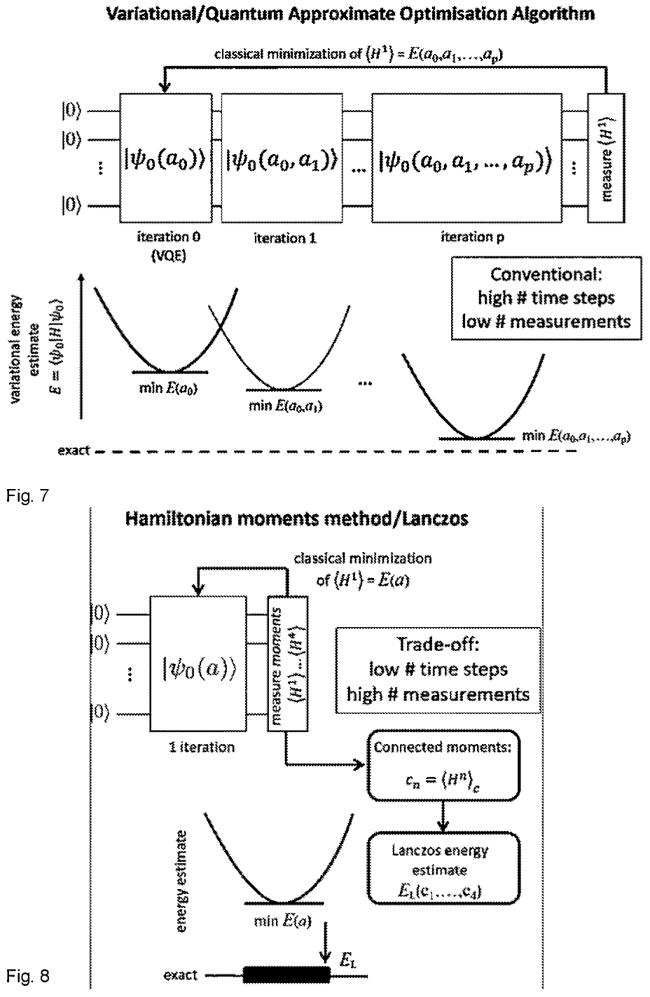

In a QC context, the problem in question is first transformed into the corresponding Hamiltonian. The determination of the system energy via a VQE approach proceeds by creating a variational trial state |ψ₀(α₀) [symbol] , w.r.t. parameters α₀, and measuring [symbol] H¹ [symbol] term by term in the Hamiltonian as shown in FIG. 7 to determine the energy E(α₀). The whole process is contained within a classical loop to choose a new value of α₀ to eventually minimise E(α₀). The quality of the VQE result is directly governed by the quality of the trial-state choice |ψ₀(α₀) [symbol] —generally this is kept as simple as possible to be created via a short depth circuit to minimize the effect of cumulative logic errors in the QC. One could systematically improve on the result using the Quantum Approximate Optimisation Algorithm (QAOA) (represented in FIG. 7 as higher iterations), but again this is at the expense of circuit depth and hence compromising the fidelity of the output.

In the moment based approach shown in FIG. 8 at first order, system 300 starts with a relatively simple short-depth trial-state as for VQE, potentially with variational parameters a. The computation of moments [symbol] H^{1 . . . 4} [symbol] then proceeds as a direct measurement of the individual operator terms in H^{1 . . . 4}. The number of Pauli strings can be large as the number of qubits in the system grows (although the increase is polynomial), however, reduction through classical pre-processing can in principle drastically reduce this scaling burden.

### The Heisenberg Model Testing Ground

The efficacy of the moments method at first order can be demonstrated via the Heisenberg Model—an interacting quantum spin system, which translates conveniently to the qubit context. The Hamiltonian over spin (qubit) degrees of freedom \vec{S}ᵢ on a lattice of link geometry { [symbol] ij [symbol] } with couplings Jᵢⱼ is given by:

$$H = {\sum }_{links 〈 ij 〉} {J}_{ij} {\vec{S}}_{i} \cdot \vec{{S}_{j}} = {\sum }_{links 〈 ij 〉} {J}_{ij}^{′} ( {X}_{i} {X}_{j} + {Y}_{i} {Y}_{j} + {Z}_{i} {Z}_{j} )$$

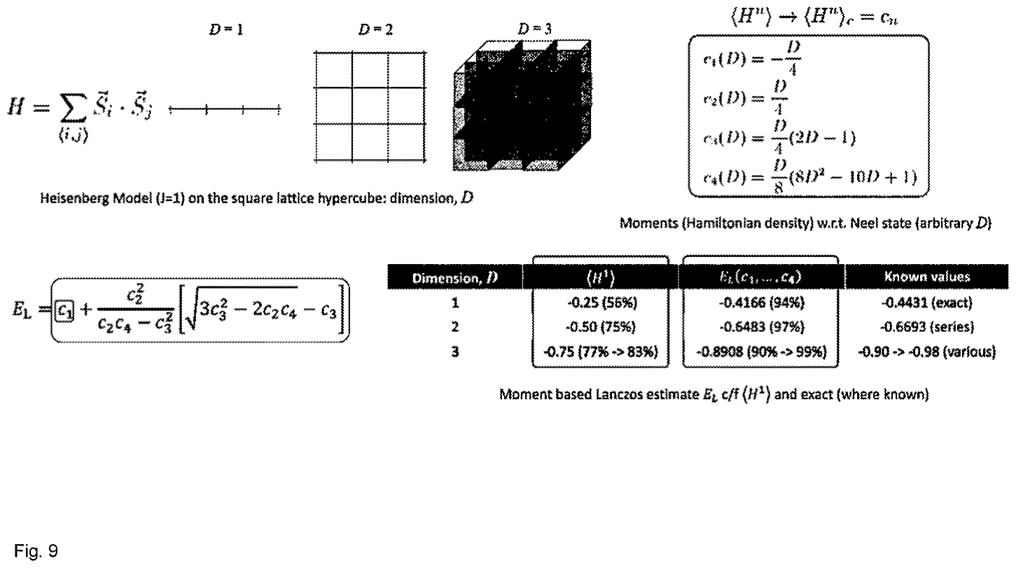

With respect to the Neel trial state (pure antiferromagnetic order) the moments can be obtained analytically for general dimension D and the first order Lanczos estimate determined (in this uniform case for the ground state energy density in the infinite lattice limit) as shown in FIG. 9. As can be seen the correction to the trial-state energy [symbol] H¹ [symbol] is significantly closer to the exact results.

Some real-world problems transformed into Hamiltonian form for QC will be highly heterogenous in couplings and contain higher order products of qubit operators. However, this is where the moment method disclosed herein is advantageous—it is not restricted to any particular coupling form.

### Testing and Verification of the Method

To demonstrate the promise of the approach in the QC context, the moments based method was applied to the Heisenberg quantum spin system for both uniform and random coupling cases. Experimental results were obtained on a series of devices for a 6-site system. The comparisons show a similar distinction between the trial-state energy [symbol] H¹ [symbol] (c.50% of exact) and the Lanczos energy estimate (c.95% of exact) for both the uniform coupling system and the random coupling instance (i.e. that the uniform coupling results are statistically significant). A similar level of improvement of the moments based Lanczos estimate over [symbol] H¹ [symbol] was found to persist when a variational parameter was introduced. The spread of results across the different devices and overall runs is indicative of the inherent relative sensitivity to device processing errors—with a tighter spread of the estimates across the devices/runs we see an indication of the relative robustness of the moment method to device errors/noise. This somewhat surprising result may be due to the binomial transformation to connected moments effectively cancelling out noise in the results.

### Higher-Order in the Moment Expansion of the Lanczos Estimates and the Infimum Theorem

We have so far focused on the Lanczos first order expansion (including moments to 4th order) because in this case the estimate E_{L} can be obtained analytically for the general case. The next level corrections based on higher moment orders can be obtained through the infimum theorem—as shown below:

Higher-order expansion of Lanczos elements:

$$α ( 𝓏 ) ≡ {c}_{1} + 𝓏 [ \frac{{c}_{3}}{{c}_{2}} ] + {𝓏}^{2} [ \frac{3 {c}_{3}^{3} - 4 {c}_{2} {c}_{3} {c}_{4} + {c}_{2}^{2} {c}_{5}}{4 {c}_{2}^{4}} ] + O ( {𝓏}^{3} ) ,$$

$${β}^{2} ≡ 𝓏 {c}_{2} + {𝓏}^{2} [ \frac{{c}_{2} {c}_{4} - {c}_{3}^{2}}{2 {c}_{2}^{2}} ] + {𝓏}^{3} [ \frac{21 {c}_{2} {c}_{3}^{2} {c}_{4} - 21 {c}_{3}^{4} - 4 {c}_{2}^{2} {c}_{4}^{2} - 6 {c}_{2}^{2} {c}_{3} {c}_{5} + {c}_{2}^{3} {c}_{6}}{12 {c}_{2}^{5}} ] + O ( {𝓏}^{4} )$$

This shows an expansion of the Lanczos elements to higher order in the connected Hamiltonian moments cₙ in a continuum form (the expansion parameter z is formally related to the Lanczos iteration index).
Higher-order Lanczos
estimates E_{L} at infimum
w.r.t. expansion parameter z

$${E}_{L} = {inf}_{𝓏 > 0} [ α ( 𝓏 ) - 2 β ( 𝓏 ) ]$$

From the infimum theorem one can obtain the Lanczos estimate of the ground state energy at any order in the moment expansion.

### Expanding the Class of Problems Via “Zero-Quantum” Extrapolation

The moment method is specifically geared to problems where the Hamiltonian is quantum and the moments themselves encapsulate dynamical corrections. For problems where the Hamiltonian is purely classical (e.g. QUBO ZZ problems) one can add-in quantum mechanical terms (“quantum spark”) which may be single or multiple qubit terms promoting tunneling to lower energy configurations, controlled by a parameter or set of parameters with respect to which results are extrapolated to the zero regime of the original problem. The moments computed by the QC could be used to determine better estimates to the ground-state energy, but also to extend the application—an excited state (by designing the trial state accordingly), and/or the configuration of the state in question (more general applicability). The quantum spark may be designed to represent a dynamical quantity to be determined (expectation value) with respect to the problem in question.

While examples show the case for problems cast into a purely quantum form (e.g. for chemistry), for optimisation that do not map to a purely quantum Hamiltonian, system 300 can force it with “quantum spark” parameter(s) λ (which may represent a set of parameters), and repeat as it reduces λ to zero. This extends the applicability of the proposed system to further categories of optimisation problems.

### Example Flow

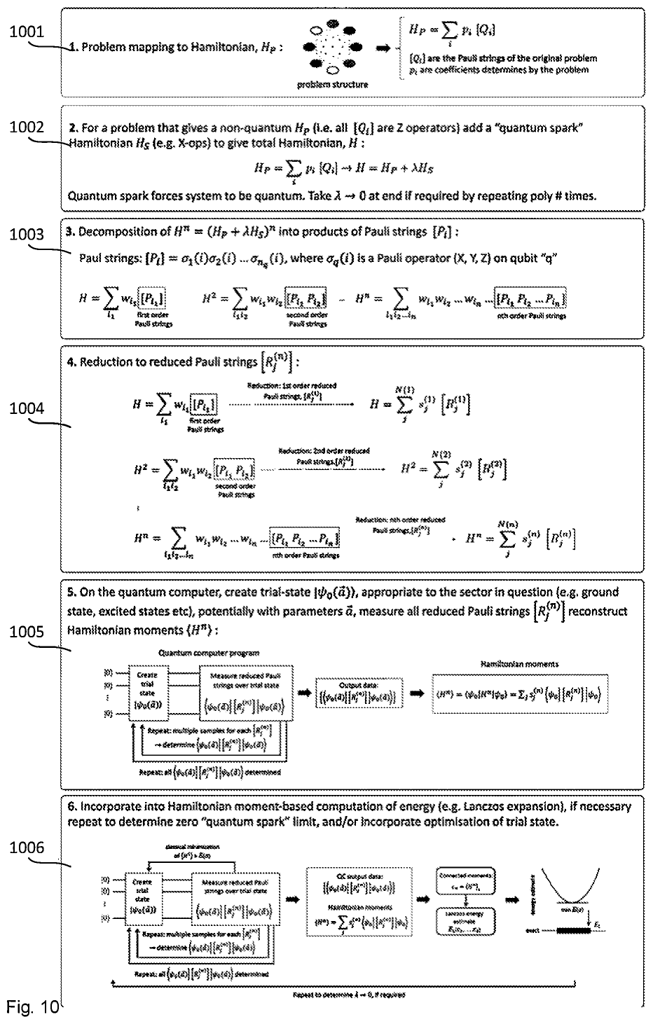

FIG. 10 illustrates an example flow of the method disclosed herein for estimating a solution to a problem represented by a Hamiltonian on a quantum computer. At 1001 the problem to be solved is mapped to a Hamiltonian. This Hamiltonian is represented as a combination of multiple first order Pauli strings. Here, this is a weighted sum of Pauli strings.

At 1002, a “quantum spark” λ (or multiple instances of such parameters) is added if the problem gives a non-quantum Hamiltonian, which forces the system to be quantum. If the problem is already quantum, this step can be skipped. The quantum spark may also be introduced for the purposes of determining a dynamical quantity.

At 1003, the higher order moments of H are represented as weighted sums of products of Pauli strings, which means the Hamiltonian and its exponentiated forms are now represented as a weighted sum of multiple first order Pauli strings and multiple, more than first order Pauli strings.

Products of Pauli strings can be reduced at 1004. Again, this step is optional. At 1004 the higher order product Pauli strings are reduced to “reduced Pauli strings” R over the commensurate number of qubits in the quantum computer.

At 1005, the actual measurement of the qubit values takes place in order to measure all reduced Pauli strings. This involves the repeated creation (i.e. initialisation) of the trial state and measurement of the qubits as an evolution of the trial state and corresponding to the first and higher order reduced Pauli strings. This repeated measurement results in multiple samples, which are then used to determine estimates for the Pauli strings by calculating expected values of the samples with respect to the trial state in question. These estimates then enable the calculation of a solution.

At 1006, the trial state is updated and the measuring steps are repeated to iteratively minimise the calculated energy as represented by the moments

### Results

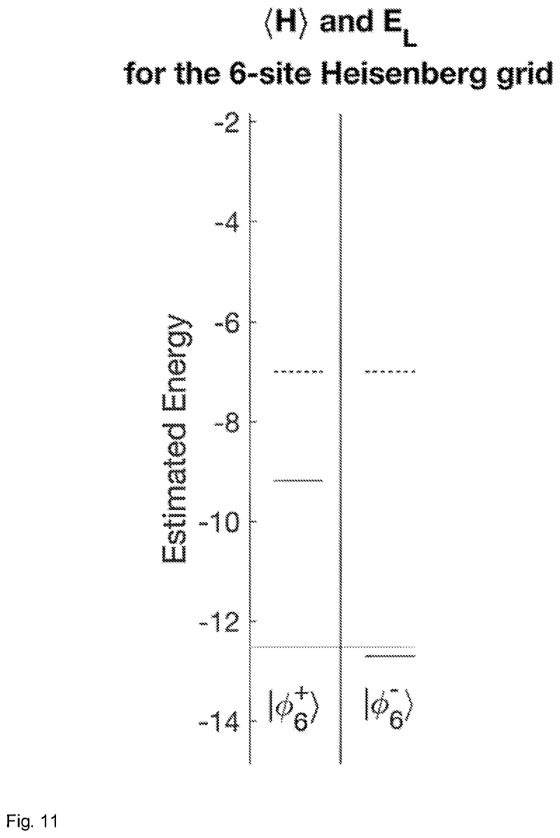

FIG. 11 illustrates results of the method described above for a 6-site Heisenberg grid,. The solid lines represent results from system 300 and the dashed lines represent results of the prior art calculation of <H> alone. It can be seen that the solid lines are significantly closer to the exact result of −12.5.

### Methods

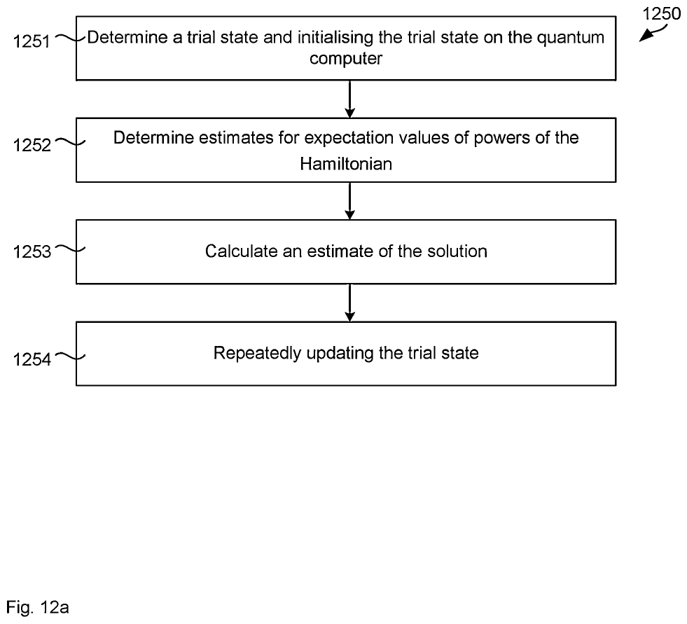

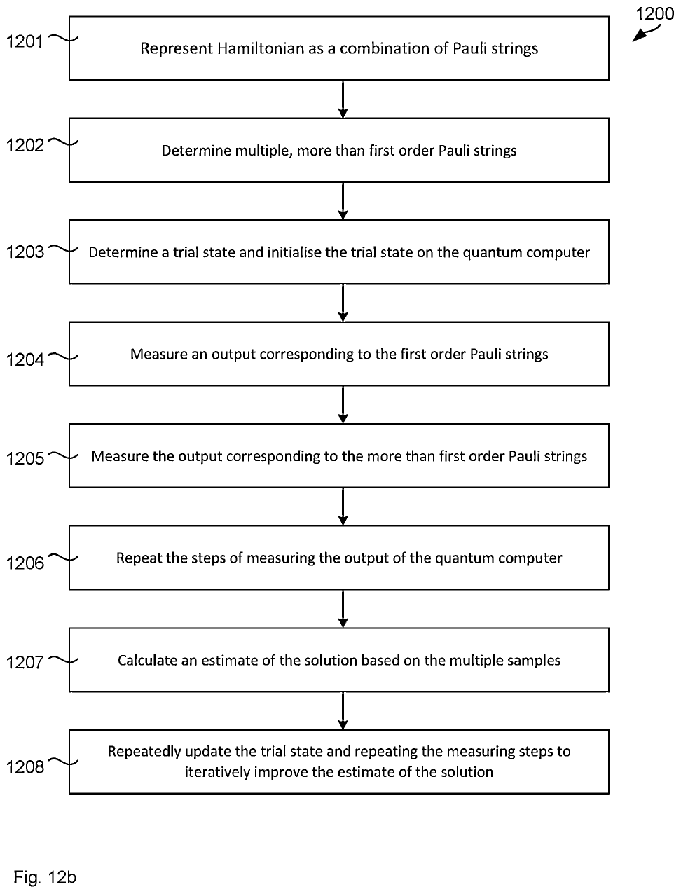

FIG. 12a illustrates a method 1250 for estimating a solution to a problem represented by a Hamiltonian on a quantum computer. The steps of method 1250 correspond to the steps described above. Method 1250 may be performed by classical computer 102 and as such, may be implemented in source code and compiled into computer readable instructions. These instructions are stored on a non-transitory, computer readable medium and cause computer 102 to perform method 1250.

More specifically, computer 102 determines 1251 a trial state and initialises the trial state on the quantum computer. Then, computer 102 determines 1252 estimates for expectation values of powers of the Hamiltonian based on multiple samples measured from the quantum computer encoding the trial state and calculates 1253 an estimate of the solution based on the estimates for the expectation values for the trial state. Next, computer 102 repeatedly updates 1254 the trial state and repeats the measuring steps to iteratively improve the estimate of the solution.

In essence, method 1250 is an expansion of the VQE in the sense that method 1250 considers powers of the Hamiltonian instead of just the Hamiltonian itself. As a result, the estimate is more accurate.

A particular example implementation, based on higher order Pauli strings is provided in FIG. 12b, which illustrates a method 1200 for estimating a solution to a problem represented by a Hamiltonian on a quantum computer. The steps of method 1200 correspond to the steps described above. Again, method 1200 may be performed by classical computer 102 and as such, may be implemented in source code and compiled into computer readable instructions. These instructions are stored on a non-transitory, computer readable medium and cause computer 102 to perform method 1200. In particular, computer 102 represents 1201 the Hamiltonian as a combination of multiple first order Pauli strings according to

$$H = {\sum }_{i} {w}_{i} {P}_{i} .$$

Computer 102 then determines 1202 multiple, more than first order Pauli strings PᵢPⱼ. Computer 102 then determines 1203 a trial state and initialising the trial state on the quantum computer. Computer 102 then measures 1204 an output corresponding to the first order Pauli strings of the quantum computer as an evolution of the trial state, to obtain <H> from the weighted sum of the measurements of first order Pauli strings. Computer 102 also measures 1205 the output corresponding to the multiple, more than first order Pauli strings of the quantum computer as an evolution of the trial state to obtain <Hⁿ> from the weighted sum of the measurements of more than first order Pauli strings. Computer 102 repeats 1206 the steps of measuring the output of the quantum computer to obtain multiple samples of the first order measurements and the more than first order measurements. Computer 102 then calculates 1207 an estimate of the solution based on the multiple samples and repeatedly updates 1208 the trial state, possibly optimising the trial-state with respect to the first moment <H>, and repeats the measuring steps to iteratively improve the estimate of the solution.

### Hamiltonian Problem, Operator Reduction and Scaling

As mentioned above, it is possible to improve the performance of the disclosed methods by using a reduction technique for the number of Pauli strings. The following section provides further details of this reduction technique. Consider a further non-trivial example system from quantum magnetism. The quadratic Hamiltonian (density) for q qubits is given by:

$$H = 1 q {\sum }_{〈 ij 〉} ( {J}_{ij}^{〈 x )} {X}_{i} {X}_{j} + {J}_{ij}^{〈 y )} {Y}_{i} {Y}_{j} + {J}_{ij}^{( 𝓏 )} {Z}_{i} {Z}_{j} ) ,$$

where the sum is over a problem graph defined by the vertices (qubits) i=1 . . . q, edges connecting qubits { [symbol] i j [symbol] }, and couplings J(s) along each edge (s=x, y, z). Here, the system considers nearest-neighbour 2D lattices with free-boundary conditions. The uniform coupling case Jᵢⱼ^{(x)}=Jᵢⱼ^{(y)}=Jᵢⱼ^{(z)} is the 2D Heisenberg model

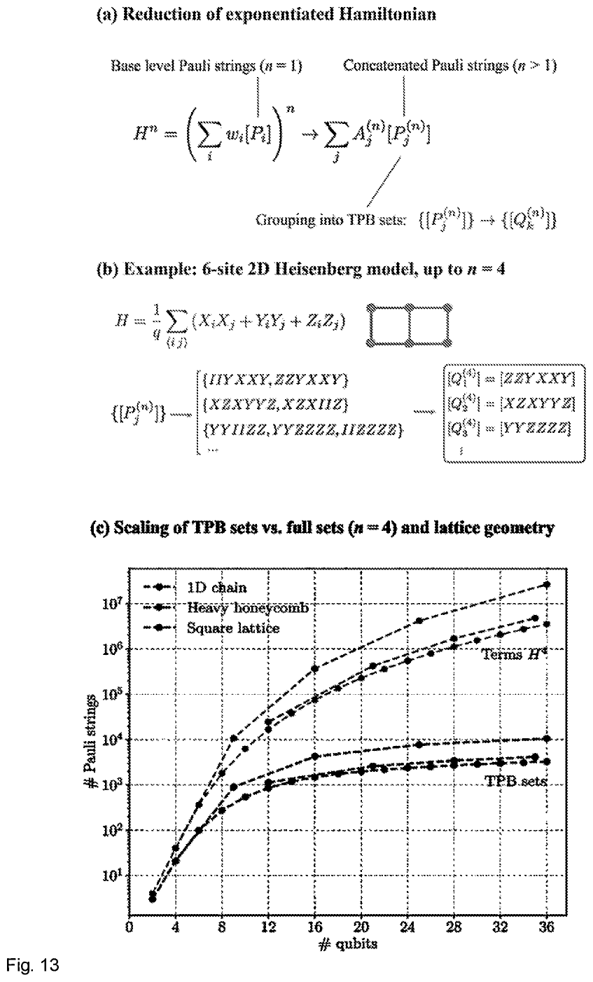

This disclosure provides detail relating to the Hamiltonian exponentiation and the scaling of the effective number of Pauli strings for measurement. Initially, the method concatenates and compresses products of Pauli strings at each level of Hⁿ:

$${H}^{n} = {( {\sum }_{i} {w}_{i} [ {P}_{i} ] )}^{n} {⇒}^{concatenation} {\sum }_{j} {A}_{j}^{( n )} [ {P}_{j}^{( n )} ] ,$$

where the [Pⱼ⁽ⁿ⁾] are q-length Pauli strings resulting from the product reductions, and Aⱼ⁽ⁿ⁾ are the resulting weights. Naive counting suggests the number of Pauli strings in the expressions corresponding to powers of the Hamiltonian may increase exponentially with n in some cases. However, by exploiting the properties of the Pauli matrices and their commutation relations, the number of strings required for measurement can be drastically reduced by finding tensor product basis (TPB) sets [Qₖ] of Pauli strings that mutually qubit-wise commute (QWC), see A. Kandala et al, “Hardware-efficient variational quantum eigensolver for small molecules and quantum magnets”, Nature 549, 242 (2017), which is included herein by reference.. Thus, we rewrite the operator H″ in terms of the TPB sets as:

$${H}^{n} = {\sum }_{j} {A}_{j}^{〈 n )} [ {P}_{j}^{〈 n )} ] : \{ [ {P}_{f}^{〈 n )} ] \} {⇒}^{TPBg r o u p i n g} \{ [ {Q}_{k}^{〈 n )} ] \} ,$$

where products of Hamiltonian-level strings [Pⱼ⁽ⁿ⁾] in Hⁿ are grouped to form TPB sets of QWC Pauli strings, [Qₖ⁽ⁿ⁾], which are labeled by the string [Qₖ⁽ⁿ⁾] itself (see FIGS. 13a and 13b. Measurement is only carried out over the [Qₖ⁽ⁿ⁾], reducing the overall measurement burden accordingly.

Finding the optimal TPB sets [Qₖ(n)] can be mapped to a minimum clique cover problem for the equivalent graph and solved heuristically. Here the method employs an identity-operator sorting algorithm. Pauli strings are sorted into a growing collection of TPB sets [Qₖ(n)], starting with the Pauli strings which have the lowest number of identities. Each [Qₖ(n)] is created when a Pauli string does not QWC with any other [Qₖ(n)] in the collection. When a Pauli string P does QWC with an existing [Qₖ(n)], it is assigned to the corresponding TPB set, and identities in the string [Qₖ(n)] change to one of X,Y,Z accordingly to ensure that any new Pauli string added to the set is QWC with P. The final TPB sets [Qₖ⁽ⁿ⁾] of Pauli strings to be measured depends on the underlying problem graph { [symbol] ij [symbol] }. To show this, we perform the reduction process for three types of graphs—linear, heavy-honeycomb, and square lattice—for systems defined on up to 36 qubits. In FIG. 13c we plot the growth with the number of qubits, q, for naive term counting. The dramatic effect of the TPB grouping process on the scaling is evident—for a given q, the number of Pauli strings to be measured drops by several orders of magnitude with sub-linear scaling in q. The qubit-wise measurements of each of the concatenated strings [Pⱼ⁽ⁿ⁾] in the TPB set [Qₖ⁽ⁿ⁾] are reconstructed from the qubit-wise measurements of the string [Qₖ⁽ⁿ⁾]. Repeated measurements are summed to produce expectation values of the Pauli strings [Pⱼ⁽ⁿ⁾] from which the moments [symbol] Hⁿ [symbol] are assembled.

### Results

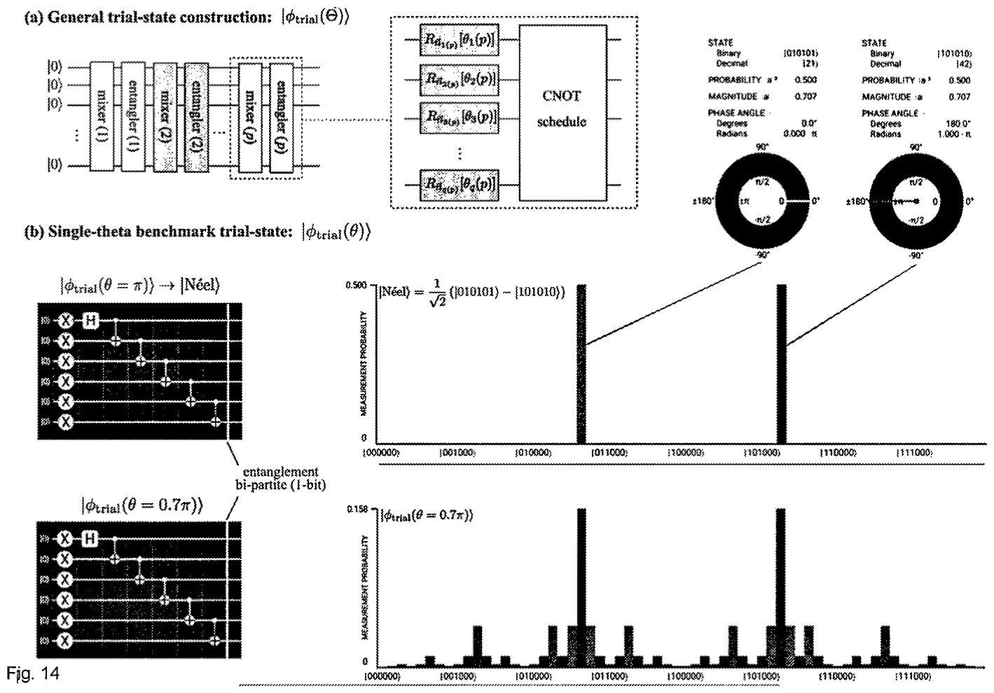

As a first application of the method we consider the case of the 2D Heisenberg model defined on a 2×3 lattice with uniform coupling J(ᵢⱼ^{(s)}=1. The first few TPB sets produced by the grouping algorithm at order H⁴ are shown in FIG. 13b. Following a simplified version of the VQE construction FIG. 14 (a), we define a trial-state in single parameter form, |ϕₜᵣᵢₐₗ(θ) [symbol] , as shown in FIG. 14(b). This choice of trial-state includes the antiferromagnetic Néel state at θ=π. Away from θ=π the full set of 2^{q} states are engaged (e.g. θ=0.7π shown). While the model itself is defined on a 2D lattice, we meet the challenge of restricted qubit array connectivity by defining the trial-state over a 1D array of qubits.

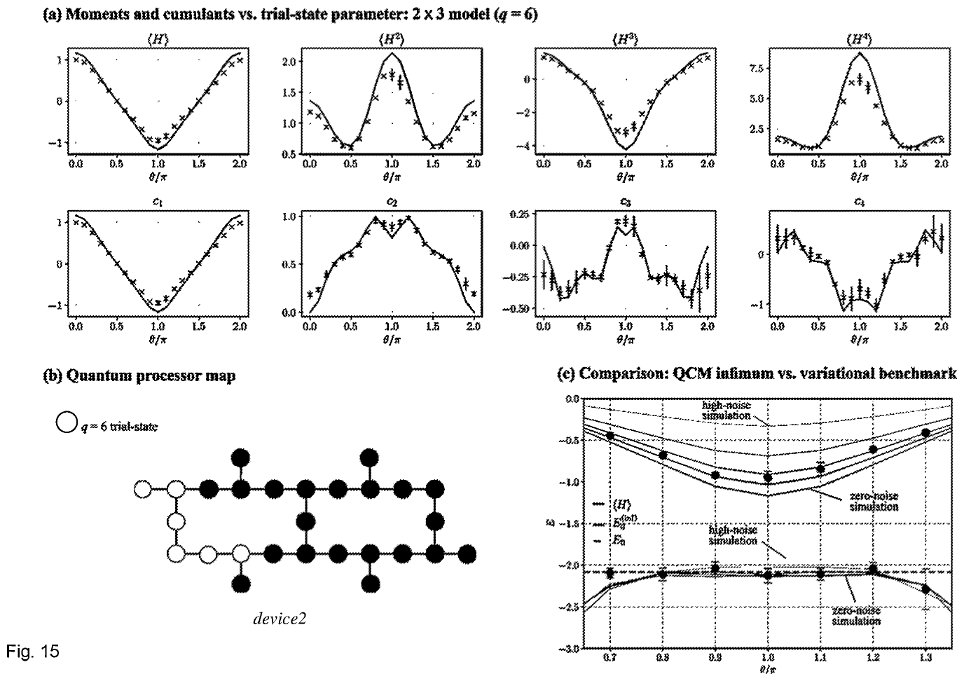

Results shown in FIG. 15 correspond to the QCM algorithm run a physical quantum processor—the qubits used are indicated on the device map. Comparison simulations were run on the Qiskit QASM simulator. The default error model used incorporates depolarizing error and readout error to match those of the available devices. The single-qubit depolarizing error rate is set to 0.001, and the two-qubit depolarizing error rate is set to 0.01. For readout errors we set Prob(0|1)=0.03 and Prob(1|0)=0.015. We have plotted, as a function of the trial-state parameter θ, the moments [symbol] Hⁿ [symbol] , and associated cumulants cₙ, assembled from the QC measurements of the TPB sets {[Qₖ⁽ⁿ⁾]} up to ₘₐₓ=4. Quantum calculations were carried out using 5×1024 shots per expectation value. It is noted that these are raw results with no attempt at error mitigation or improved sampling. Compared to the exact/simulation results (solid lines), the moments computed on the quantum computer system are surprisingly free of shot noise, with deviations largely due to the device errors. The cumulants have higher statistical noise, as expected given their composition in terms of the moments. In FIG. 15(c) we plot the infimum estimates E₀^{(inf)} obtained from the device runs together with variational results on [symbol] H [symbol] _{θ} and simulations carried out for different noise levels (zero to 8× device default error model). We make the following observations:

(i) The infimum estimate significantly improves on the variational result for the same trial-state;

(ii) Despite the classical manipulation of measured quantities to assemble [symbol] Hⁿ [symbol] and cₙ, the overall statistical noise in the final QCM infimum results appears to be not too much greater than the variational results, and certainly much less than their difference;

(iii) The quality of the infimum estimate derived from the trial-state on the 1D qubit array persists for a range of values of the trial-state parameter either side of the variational minimum (θ=π);

(iv) The simulations indicate the infimum estimate is more robust to device noise than the variational calculation on [symbol] H [symbol] _{θ}.

To test these observations we move on to larger and more complex instances of the 2D model. The 1D trial-state form |ϕₜᵣᵢₐₗ(θ) [symbol] is retained, but the model is generalised to the case of random couplings, {Jᵢⱼ^{(x,y,z)}}. We note another important feature of the QCM approach—once the Pauli string reduction and measurements have been carried out for a particular problem graph, one need not repeat when the couplings in the problem Hamiltonian are changed. In effect, one only needs to run the moments computation once on the quantum computer—the infimum estimates for an arbitrary large ensemble of random instances can be computed efficiently using classical resources post-facto by recycling the quantum computed moments output. For problem instances up to 4×4 we compute and compare with exact results, however, at 5×5 the Hilbert space dimension of the problem is O(10⁷) and begins to challenge convenient classical computation. As a reference, we compare with the 2D Heisenberg model case with uniform couplings for which the ground-state can be determined numerically (Note the infinite lattice limit value is E₀=−2.676 in our qubit notation).

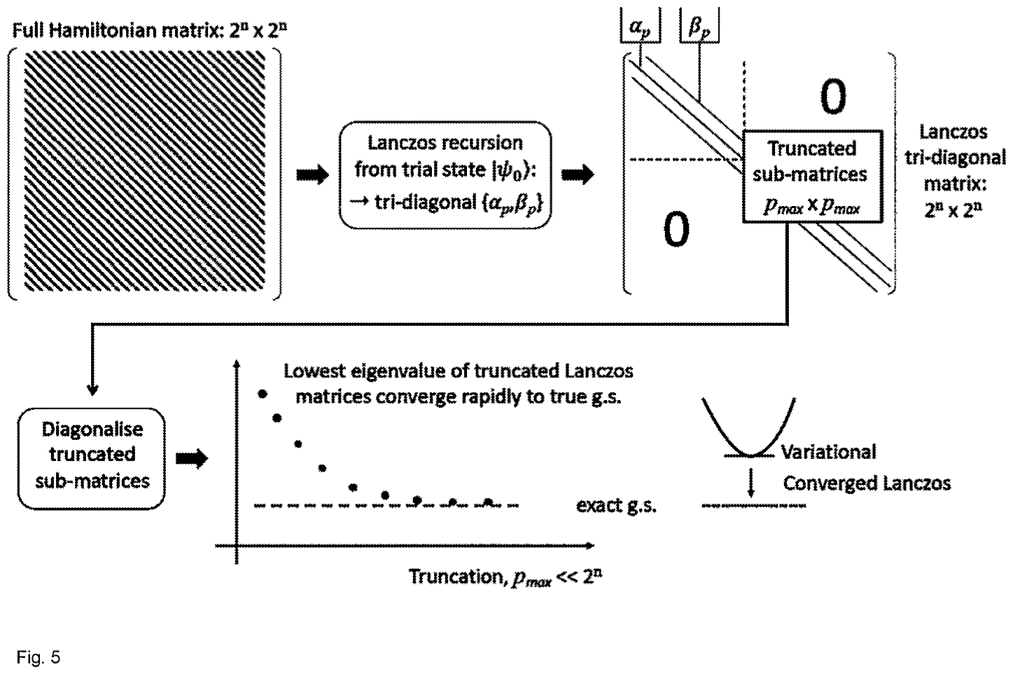

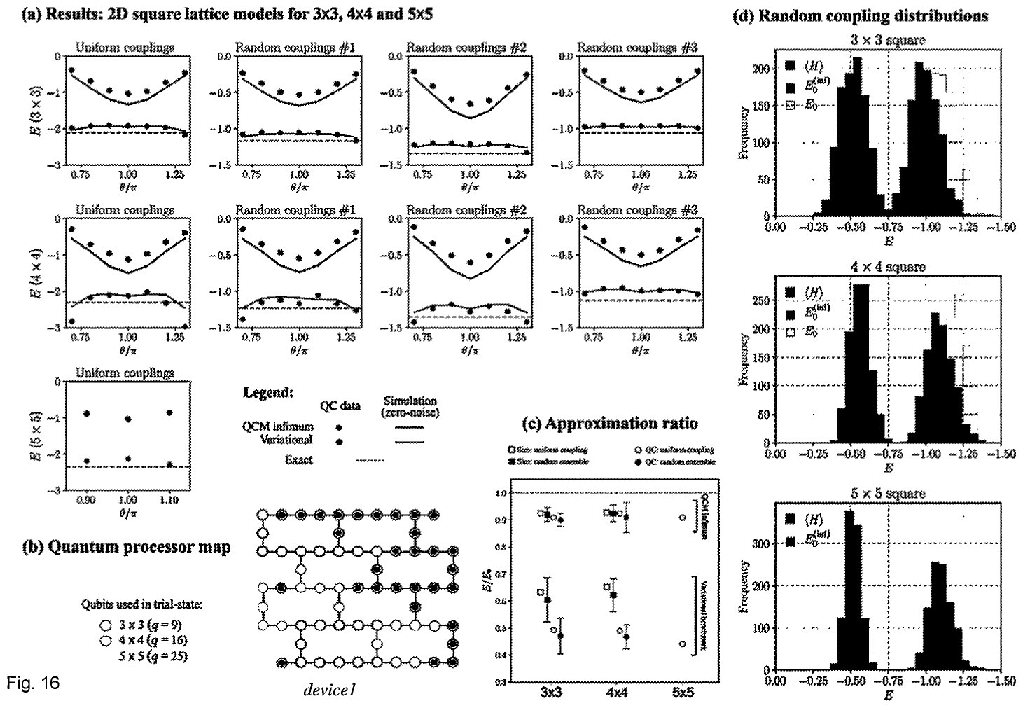

In FIG. 16(a) we show the results for square lattices 3×3, 4×4, and 5×5 (8192 shots per data point) with the trial-state implemented on qubit chains as shown on the IBM Quantum device ibmq_manhattan FIG. 5(b). Random coupling instances correspond to choosing Jᵢⱼ^{(x,y,z)}∈[0.1) (three decimal places). Across the board, the QCM infimum results improve on the variational benchmark and are remarkably close to the exact results given the 1D restriction of |ϕₜᵣᵢₐₗ(θ) [symbol] . The data is accompanied by zero-noise simulations, which clearly show that while the variational data points obtained with device noise consistently move away from the true ground-state energy, the QCM infimum estimates around the Néel point are remarkably inert and maintain the robustness to both noise and change in the trial-state as shown in the q=6 results. In FIG. 5(c) we plot the approximation ratio with respect to the exact result. The difference between the QCM results and the variational benchmark is clear—device and shot noise are well under control for the QCM infimum estimates. For the largest 5×5 instance, the approximation ratio with respect to the exact value (uniform 2D Heisenberg model) for the QCM infimum estimates is 91%. The recycling of the one-time quantum output also works well, despite the assembly process for [symbol] Hⁿ [symbol] and cₙ, providing an ensemble of results over random coupling instances—the ensemble distributions (10³ instances) are shown in FIG. 16(d).

The following references are incorporated herein in their entirety as the provide further information on the Lanczos method and plaquette expansion:

L. Hollenberg, Plaquette expansion in lattice Hamiltonian models, Phys. Rev. D, 1993, doi:10.1103/PhysRevD.47.1640

L. Hollenberg & N. S. Witte, A General Non-Perturbative Estimate of the Energy Density of Lattice Hamiltonians, Phys. Rev. D, 1994, doi:10.1103/PhysRevD.50.3382

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for estimating a solution to a problem represented by a Hamiltonian on a quantum computer having a quantum state, the method comprising:
determining a trial state and evolving the quantum state of the quantum computer based on the trial state;
determining estimates for expectation values with respect to the trial state of powers of the Hamiltonian based on measurements from the quantum computer;
calculating an estimate of the solution based on the estimates for the expectation values of powers of the Hamiltonian for the trial state; and
repeatedly updating the trial state and repeating the measuring steps to iteratively improve the estimate of the solution.

2. The method of claim 1, wherein the solution to the problem is an energy state of a physical system represented by the Hamiltonian on a quantum computer.

3. (canceled)

4. The method of claim 1, wherein the solution to the problem is a configuration of the physical system.

5. (canceled)

6. (canceled)

7. The method of claim 1, wherein calculating the estimate of the solution comprises calculating more than first order moments of the Hamiltonian and calculating the estimate of the solution based on the more than first order moments.

8. The method of claim 7, wherein calculating the estimate of the solution is based on moments or cumulants that represent connected moments.

9. The method of claim wherein the moments or cumulants correspond to values required for a Lanczos method.

10. The method of claim 9, wherein the Lanczos method is based on parameters of an approximate representation of the Hamiltonian on a basis of the trial state and calculating the estimate of the solution is based on the parameters of the approximate representation of the Hamiltonian.

11. The method of claim 10, wherein the approximate representation of the Hamiltonian comprises a tri-diagonal matrix.

12. The method of claim 8, wherein the cumulants are based on a binomial transformation of the more than first order moments.

13. (canceled)

14. The method of claim 1, wherein the trial state comprises one or more adjustable parameters and updating the trial state comprises updating the one or more adjustable parameters to iteratively improve the estimate of the solution.

15. The method of claim 1, further comprising:
representing the Hamiltonian as a decomposition of multiple first order operational components and the powers of the Hamiltonian as a decomposition of multiple, more than first order operational components.

16. The method of claim 1, further comprising:
representing the Hamiltonian as a combination of multiple first order Pauli strings and the powers of the Hamiltonian as multiple, more than first order Pauli strings;
measuring an output corresponding to the multiple first order Pauli strings of the quantum computer as an evolution of the trial state, to obtain first order measurements;
measuring the output corresponding to the multiple, more than first order Pauli strings of the quantum computer as an evolution of the trial state to obtain multiple, more than first order measurements; and
repeating the steps of measuring the output of the quantum computer to obtain the multiple samples of the first order measurements and the more than first order measurements for determining the estimates for the expectation values.

17. The method of claim 16, wherein determining the multiple, more than first order Pauli strings comprises determining multiples of the multiple first order Pauli strings.

18. The method of claim 16, wherein measuring the output corresponding to the multiple, more than first order Pauli strings comprises applying quantum operations corresponding to the multiple, more than first order Pauli strings to the quantum computer.

19. The method of claim 16, wherein determining the multiple, more than first order Pauli strings comprises combining elements in the multiple, more than first order Pauli strings into equivalent elements to reduce the multiple, more than first order Pauli strings.

20. The method of claim 19, wherein combining the elements comprises determining tensor product basis sets of Pauli strings that mutually qubit-wise commute.

21. The method of claim 20, wherein measuring the output corresponding to the multiple, more than first order Pauli strings comprises measuring only the tensor product basis sets.

22. The method of claim 16, wherein the Hamiltonian is associated with a parameter or set of parameters that is included in the multiple first order Pauli strings and the multiple, more than first order Pauli strings; and
the method comprises taking the appropriate limit of the parameter or set of parameters.

23. The method of claim 22, wherein the parameter or set of parameters is associated with explicit quantum-mechanical term or terms representing a dynamical quantity to be determined with respect to the problem.

24. A system for estimating a solution to a problem represented by a Hamiltonian on a digital quantum computer, the system comprising:
multiple qubits;
a trial state controller to set the multiple qubits into a trial state;
a read-out component to measure a quantum state of the qubits; and
a classical computer to control the trial state controller and the read-out component, the classical computer being configured to:
determine a trial state and initialising the trial state on the quantum computer;
determine estimates for expectation values of powers of the Hamiltonian based on multiple samples measured from the quantum computer encoding the trial state;
calculate an estimate of the solution based on the estimates for the expectation values of powers of the Hamiltonian for the trial state; and
repeatedly update the trial state and repeat the measuring steps to iteratively improve the estimate of the solution.
